SEQUENCE LISTING (8 sequences)

SEQ ID NO: 1 - DNA, 74 bp, Artificial sequence
  Oligo 1
gccgatatcggacaacggccgaactgggaaggcgagactgaccgaccgataagctagaacagagagacaacaac

SEQ ID NO: 2 - RNA, 38 nt, Artificial sequence
  Oligo 2
uaggcgucggugacaaacggccagcguuguugucucuc

SEQ ID NO: 3 - DNA, 25 bp, Artificial sequence
  Forward primer
ggacaacggccgaactgggaaggcg

SEQ ID NO: 4 - DNA, 25 bp, Artificial sequence
  Reverse primer
taggcgtcggtgacaaacggccagc

SEQ ID NO: 5 - DNA, 30 bp, Artificial sequence
  Detection probe
actgaccgaccgataagctagaacagagag

SEQ ID NO: 6 - DNA, 99 bp, Artificial sequence
  Internal control
gccgatatcggacaacggccgaactgggaaggcgagatcagcaggccacacgttaaagacagagagacaacaacgctggccgtttgtcaccgacgccta

SEQ ID NO: 7 - DNA, 25 bp, Artificial sequence
  Probe
atcagcaggccacacgttaaagaca

SEQ ID NO: 8 - DNA, 99 bp, Artificial sequence
  Polymerisation product
gccgatatcggacaacggccgaactgggaaggcgagactgaccgaccgataagctagaacagagagacaacaacgctggccgtttgtcaccgacgccta

(19) United States
(12) Patent Application Publication
(10) Pub. No.: US 2016/0083776 A1
(43) Pub. Date: Mar. 24, 2016

(54) Methods for Measuring Polymerase Activity Useful for Sensitive, Quantitative Measurements of Any Polymerase Extension Activity and for Determining the Presence of Viable Cells

(71) Applicant: Zeus Scientific, Inc., Perkasie, PA (US)

(72) Inventors: Shawn Mark O'Hara, Raritan, NJ (US); Daniel Zweitzig, Raritan, NJ (US)

(21) Appl. No.: 14/391,758

(22) PCT Filed: Apr. 12, 2013
(86) PCT No.: PCT/US2013/036264

§ 371 Date: Oct. 10, 2014

Related U.S. Application Data
(60) Provisional application No. 61/623,114, filed on Apr. 12, 2012.

(51) Int. Cl. C12Q 1/48

(52) U.S. Cl. CPC C12Q 1/485; G01N 2333/9126; G01N 2333/9128

(57) ABSTRACT

A novel, highly sensitive, quantitative and rapid DPE-PCR assay is disclosed that can be used to enumerate prokaryotic cells when presenting a purified or selected cell type and that has the capability to reproducibly measure DNA polymerase extension activity from less than 10 cfu of bacteria via coupling to bead lysis. Also disclosed is the potential for the DPE-PCR assay of the invention to universally detect microbes by testing a panel of microorganisms comprised of gram-negative bacteria, gram-positive bacteria and Candida species. Furthermore, it is disclosed that the DPE-PCR assay of the invention can be used to assess bacterial cell viability, provided via the reproducibly strong correlation between DNA polymerase extension activity and proliferation as indicated by the presence of cfu. It is believed that the disclosed assay of the invention can be a useful quantitative tool for a wide range of testing applications within pharmaceutical, environmental, food and clinical settings.

### CROSS REFERENCE TO RELATED APPLICATION

This application is a non-provisional application, which incorporates by reference herein and claims priority of U.S. Provisional Application No. 61/623,114, filed Apr. 12, 2012.

### BACKGROUND OF THE INVENTION

The reference numbers used in this section and throughout this disclosure refer to the documents set forth in the “References” section herein.

DNA polymerase activity is indispensable for genome replication and organism propagation across all biological domains (1-3). Procaryots contain five different types of DNA polymerases but mammalian cells contain fifteen distinct cellular DNA polymerases but only four of these are devoted to DNA replication, whereas the rest are devoted to DNA repair and specialized DNA synthetic processes that contribute substantially to the maintenance of genetic integrity. Although most of these enzymes are involved in nuclear DNA repair and replication, DNA polymerase gamma (Polg) remains the only DNA polymerase found in mitochondria (Hum. Mol. Genet. (1 Jul. 2005) 14(13): 1775-1783). Since its initial characterization (4), the ability to harness DNA polymerase activity in vitro has become a fundamental tool in the field of molecular biology research (5). Above and beyond its established importance in research, in vitro measurement of DNA polymerase activity potentially offers numerous useful applications within the pharmaceutical and clinical setting. For instance, since bacterial DNA polymerase is actively being targeted for development of novel antimicrobial agents (6, 7), a rapid and sensitive assay capable of measuring DNA polymerase activity is desirable. Also, loss or gain of DNA polymerase activity is intimately involved in human disease. For example, emerging links between DNA polymerase activity and genetic aberrations are designating the enzyme as a target for anti-cancer therapies (8, 9). Deficiencies in DNA polymerase activity have also been linked to mitochondrial disorders (10). Furthermore, measurement of DNA polymerase activity has the potential to be used as a rapid and sensitive diagnostic tool, capable of detecting virtually any organism harboring active DNA polymerase within a given environmental or biological matrix where sterility is expected.

The most common method used to measure DNA polymerase activity in vitro depends upon incorporation of radiolabeled nucleotides (11). However, routine use of such DNA polymerase assays is undesirable due to the inherent risks and restrictions associated with radioisotopes. Consequently, over the past few decades numerous non-radioactive in vitro polymerase assays have been developed. Some rely upon the measurement of fluorescence generated by DNA polymerase-mediated release of single stranded binding protein (12) or binding of PicoGreen™ to double stranded DNA (13,14). Other methods rely on microplate coupling and detection of fluorescently-labeled nucleotides (15). More recently, molecular beacon-based (16) and electrochemical-based (17) DNA polymerase assays have been developed. Despite successfully averting the use of radioactivity, the above assays are limited by either poor sensitivity, a small linear dynamic range of measurement or the use of purified polymerase. During the past fifty years, in vitro measurement of polymerase activity has become an essential molecular biology tool. Traditional methods used to measure polymerase activity in vitro are undesirable due to the usage of radionucleotides. Fluorescence-based polymerase assays have been developed; however, they also suffer from various limitations.

### SUMMARY OF THE INVENTION

In accordance with the present invention, the various limitations of the above-described methodologies have been sought to be addressed, and a rapid, highly sensitive and quantitative assay is provided capable of measuring polymerase extension activity from purified polymerases or directly from crude cell lysates, or subcellular organelles. When tested with purified DNA polymerase, the assay detected as little as 2×10⁻¹¹ U of enzyme (≈50 molecules), while demonstrating excellent linearity (R²=0.992). The assay was also able to detect endogenous DNA polymerase extension activity down to at least 10 colony forming units of input gram-positive or gram-negative bacteria when coupled to bead mill lysis while maintaining an R²=0.999. Furthermore, in accordance with the invention, it has been shown that DNA polymerase extension activity is an indicator of cell viability, as demonstrated by the reproducibly strong concordance between assay signal viable cell enumerations. Similarly, by selective sample cell preparation, intact mammalian cells can also be quantitated and viability assessed by DNA polymerase extension assay. Together, the novel methods of the invention described herein represent a significant advancement toward sensitive detection of potentially any cell or subcellular organelle containing active polymerases within a given sample matrix.

The present inventors have had an ongoing interest in methodologies involving enzymatic template generation and amplification (ETGA). For example, U.S. patent application Ser. No. 13/641,480, filed Oct. 16, 2012 and commonly assigned herewith, describes a novel technology related to such ETGA methodologies and uses thereof. To the extent such technology related to such ETGA methodologies of said U.S. Ser. No. 13/641,480 is not explicitly described herein and may be necessary to the full disclosure of the inventions described and claimed herein, the entire disclosure of said US patent application is hereby incorporated into this specification by reference.

Herein we describe the characterization of an improved, novel ETGA methodology based upon the measurement of DNA polymerase extension activity coupled to a quantitative PCR readout. Herein, we will refer to this assay approach generally as ETGA, or, also as DNA polymerase extension coupled polymerase chain reaction (DPE-PCR). Variations in sample preparation can and will be combined with ETGA and DEP-PCR for specific applications with specific cell or polymerase types.

### BRIEF DESCRIPTION OF THE DRAWING FIGURES

FIG. 1 shows a basic overview of the novel DPE-PCR assay provided by the present invention. DNA polymerase is incubated with a substrate consisting of pre-annealed Oligo-1 and Oligo-2. DNA polymerase extends only the 3′ end of Oligo-1 during a 20 minute incubation at 37° C. Three micro-liters of the DNA polymerase extension reaction mixture is subsequently transferred into a hot start qPCR reaction containing uracil DNA glycosylase (UDG). Prior to and during activation of Taq, UDG degrades the deoxyuridine within Oligo-2, leaving only a single stranded product derived from DNA polymerase-mediated extension of Oligo-1. After activation of Taq, PCR-based amplification is initiated via primer binding to the Oligo-1 extension product. The sequence of a competitive internal control DNA is presented. The competitive internal control is present at 40 copies within each PCR reaction. FIG. 1A shows a schematic overview of the mechanisms involved in coupling DNA polymerase extension activity to qPCR. FIG. 2A shows detection of DNA polymerase I extension activity in accordance with the present invention, achieved over a wide range of input enzyme.

FIG. 2 shows representations of sensitive detection of purified DNA polymerase using a preferred DPE-PCR assay in accordance with the present invention. A commercial source of DNA polymerase I was assayed in duplicate at 10 fold increments starting at 2×10⁻⁵ Units (U) down to 2×10⁻¹¹ U per reaction. A representative DPE-PCR curve is shown for each polymerase input level and No Input Control (NIC). A plot was constructed from n=4 data points per polymerase input level, taken from two independent experiments and linear regression analysis was performed. Triplicate reactions containing 2×10⁻⁷ U of DNA polymerase I, Klenow, Klenow (exo-) and E. coli DNA Ligase were assayed in comparison to a NIC. A representative DPE-PCR curve is presented for each of the assayed enzymes and NIC. Triplicate DPE-PCR curves are shown from corresponding DNA polymerase extension reactions containing a 50 μM [dATP, dGTP, dTTP] mixture supplemented with 50 μM of either dCTP or ddCTP. A schematic representing some of the first available sites for dCTP or ddCTP incorporation within the DNA substrate is presented adjacent to the DPE-PCR curves. FIG. 2B shows a regression analysis having a strong positive linear correlation (R²=0.992) between the DPE-PCR cycle threshold (Ct) values and units of input commercial DNA polymerase I after graphing data from two independent limit of detection experiments. FIG. 2C shows that both Klenow and Klenow exo—were detected in accordance with the invention at similar levels when compared to wild type DNA polymerase I, providing evidence that the DPE-PCR assay signal is derived from DNA polymerase-dependent extension and not intrinsic exonuclease activity. FIG. 2D illustrates the first possible position within the substrate that ddCTP can be incorporated by DNA polymerase.

FIG. 3 shows a schematic overview of coupling bead lysis to DPE-PCR, and illustrates a liquid sample known to contain, or suspected of containing, microbes, added to a bead mill lysis tube, disrupted and immediately transitioned into the DPE-PCR assay of the present invention.

FIG. 4 illustrates that a DPE-PCR assay in accordance with the present invention enables sensitive and quantitative detection of gram negative and gram positive bacteria via measurement of DNA polymerase extension activity in crude lysates. Decreasing amounts of E. coli cfu were spiked into bead lysis-coupled DPE-PCR. No Input Controls (NIC) were also included to monitor reagent background levels. All cfu spikes and NICs were performed in triplicate. A representative DPE-PCR curve is shown below for each level of bacterial input. Colony count plating and gsPCR were performed in an effort to obtain a better estimate of the actual cfu placed into each. A plot of E. coli DNA polymerase activity and linear regression analysis is presented. Graphs were generated using the average Ct values obtained from triplicate reactions of bacterial spikes ranging from 1×10⁵-1×10¹ input cfu. cfu titration experiments were performed for S. aureus exactly as described above for E. coli. Colony count plating. FIG. 4A, when linked with bead mill lysis, shows that the DPE-PCR assay of the invention is capable of detecting a wide dynamic range of input E. coli, down to and below 10 colony forming units (cfu) per lysis tube. In FIG. 4B, a linear regression analysis of E. coli detection is shown that was also performed down to 10 cfu of input bacteria, and showed a strong positive linear correlation between input cfu and DNA polymerase extension activity signal as indicated by an R² value of 0.999. As shown in FIG. 4C, DNA polymerase extension activity from S. aureus lysates was detected to a similar input level, and as shown in FIG. 4D, S. aureus detection was plotted down to 10 cfu of input bacteria and also showed a strong linear correlation between input cfu and DNA polymerase extension activity signal (R²=0.999).

FIG. 5 illustrates that detection of bacteria by DPE-PCR is blocked by ddCTP. 5 μL of E. coli suspension were added to bead lysis-coupled DNA polymerase assays comprised of a dNTP mix containing either 50 μM dCTP or 50 μM ddCTP. DPE-PCR curves representing E. coli-derived DNA polymerase activity is presented. Plots were generated using the average qPCR Ct values from triplicate reactions at the indicated conditions. ddCTP termination and dCTP rescue experiments were performed for S. aureus exactly as described above for E. coli. FIGS. 5A and 5B, when compared to the standard reaction mix, show that substitution of ddCTP blocked the generation of signal derived from E. coli, S. aureus cfu spikes.

FIG. 6 shows PC ETGA PCR Data generated by performance of preferred embodiments of the assay of the present invention.

### DETAILED DESCRIPTION OF PREFERRED EMBODIMENTS OF THE INVENTION

### Overview

The measurement of DNA polymerase extension activity could represent a useful tool with far reaching applications such as, but not limited to, screening candidate-polymerase inhibitors in vitro, or depending of cell selective sample preparation, detection of the presence any viable cell type (harboring active DNA polymerases) within a diverse range of sample types. If intended for these purposes, routine use of traditional polymerase assays that incorporate radiolabeled nucleotides is unattractive. Consequently, numerous non-radioactive DNA polymerase extension assays have been developed in recent decades. Despite successfully averting the use of radioactivity, current fluorescence-based DNA polymerase assays also suffer from various deficiencies. For example, detection of DNA polymerase activity via several existing non-radioactive assays is dependent upon the binding of PicoGreen™ to newly-generated double stranded DNA (13,14). If intended to analyze DNA polymerase activity from freshly lysed organisms, PicoGreen™-based assays would likely be hampered by background fluorescence via binding of PicoGreen™ to genomic DNA. Microplate-based DNA polymerase assays have also been developed (15). Decreased sensitivity of microplate-based assays can be expected for numerous reasons, including dependence upon intermediate binding of either product or substrate to a microplate and/or inefficient incorporation of modified dNTPs by DNA polymerase. More recently, real-time measurement of DNA polymerase activity via molecular beacons has been described (16). Despite improved sensitivity, direct measurement of molecular beacon fluorescence could also potentially be hindered by exposure to crude cellular lysates.

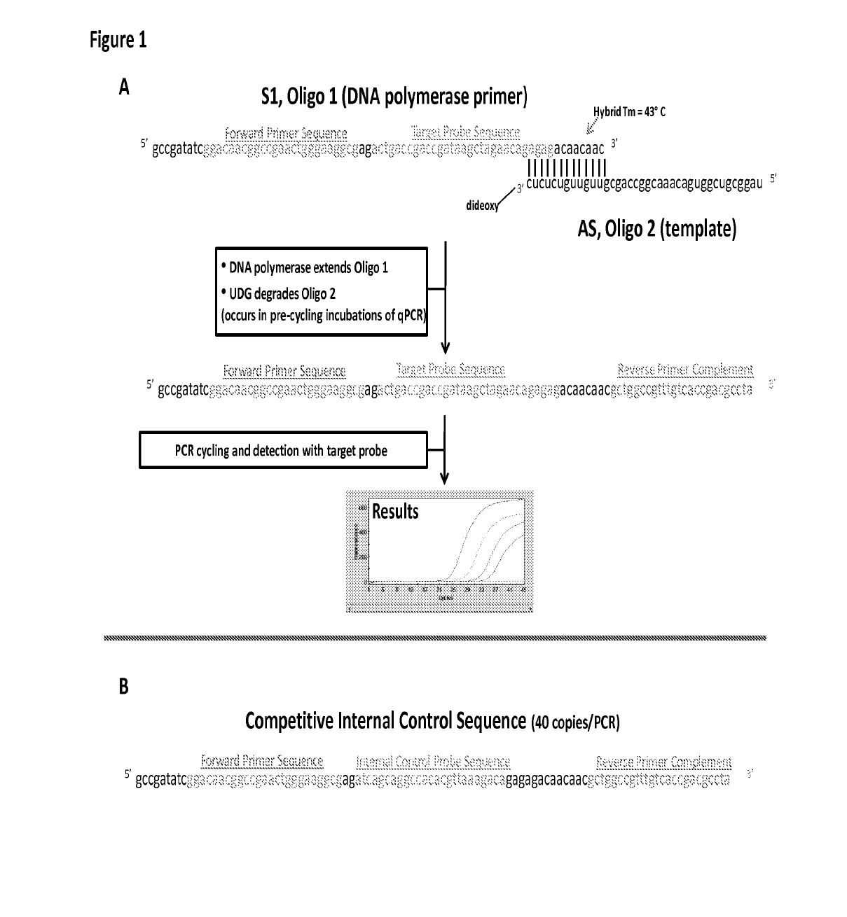

In the development of the present invention, we set out to develop a rapid, simple, highly sensitive and quantitative assay capable of measuring DNA polymerase extension activity derived from purified commercial sources or freshly lysed viable cells of any type. FIG. 1A contains a schematic overview of the mechanisms involved in coupling DNA polymerase extension activity to qPCR. Notably, Oligo 2 is eliminated by uracil DNA glycosylase (UDG) prior to and during Taq activation, thus preventing undesired Taq-dependent extension of the substrate just prior to PCR cycling. A microbial detection method linking T4 DNA ligase activity to PCR amplification has been previously reported (18), which contains similarities to our DPE-PCR assay and is another example of an ETGA methodology. However, in our hands a modified version of this method, aimed at detecting microbial-derived NAD-dependent DNA ligase activity, suffered from a lack of sensitive and universal microbial detection, leading us to the development of the improved novel DNA polymerase-based approach named DPE-PCR described herein.

### Example 1

### Sensitive and Linear Detection of Purified DNA Polymerase Extension Activity, Foundation for a Relative Quantitative Assay

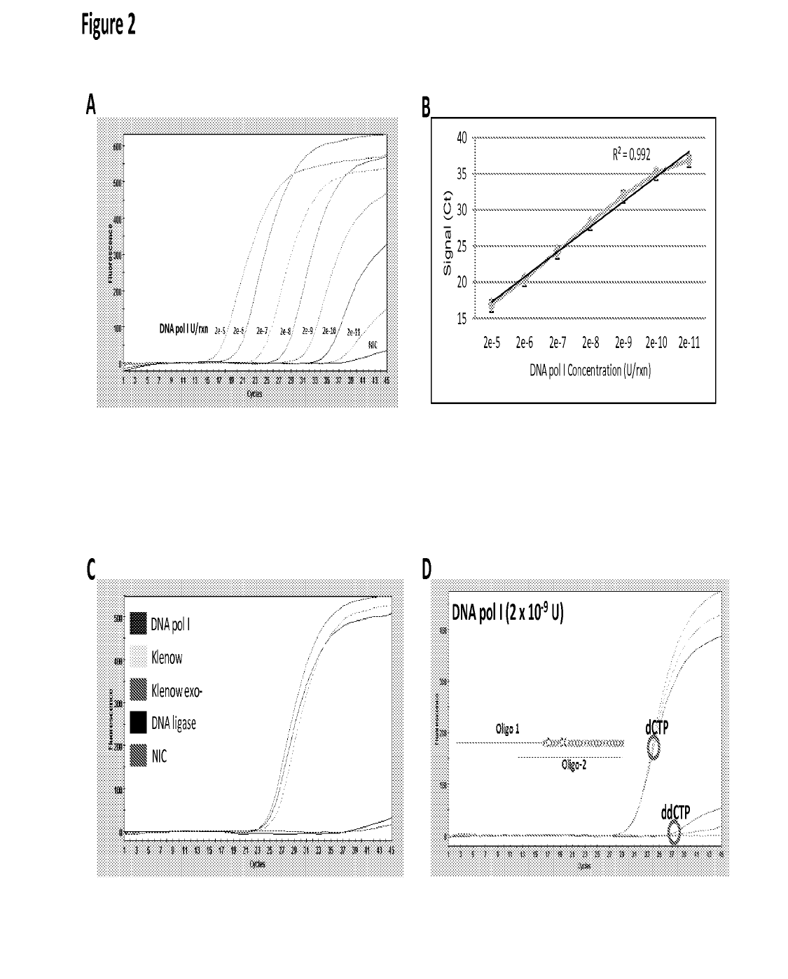

We set out to determine the approximate analytical sensitivity of the DPE-PCR assay using commercially available DNA polymerase I. In this example, DPE-PCR signals derived from decreasing amounts of DNA polymerase I were compared to parallel reactions without input DNA polymerase (Referred to hereafter as the “No Input Control” or NIC). As shown in FIG. 2A, detection of DNA polymerase I extension activity was achieved over a wide range of input enzyme. In fact, DNA polymerase I extension activity was distinguishable from the NIC down to as little as 2×10⁻¹¹ units (U) of enzyme (equivalent to approximately 50 molecules of polymerase). To our knowledge, detection of DNA polymerase extension activity at this level is unrivaled in existing DNA polymerase assays. In theory, this level of sensitivity could enable single cells to be readily detectable as microbe detection as E. coli has been reported to contain approximately 400 DNA polymerase I molecules per cell (11) similar molecule numbers per cell have been reported for mammalian DNA polymerases. Regression analysis also showed a strong positive linear correlation (R²=0.992) between the DPE-PCR cycle threshold (Ct) values and units of input commercial DNA polymerase I after graphing data from two independent limit of detection experiments (FIG. 2B). This surprisingly excellent linear relationship down to 50 DNA polymerase molecules provides the foundation for development of a reliable and robust quantitative assay for DNA polymerase molecules, intact cells and the subcellular organelles that harbor these polymerases such as nuclei and mitochondria.

After sensitivity and linearity experiments were performed, it was important to determine if the DPE-PCR assay signal was independent of intrinsic exonuclease activity. To this end, we subsequently compared signals generated by 2×10⁻⁷ U of DNA polymerase I to those generated from DNA polymerase I lacking 5′→3′ exonuclease activity (Klenow) and another version of the enzyme lacking all exonuclease activity (Klenow exo −). For additional specificity and background signal determination, E. coli DNA ligase at 2×10⁻⁷ U and a NIC were tested in parallel. As shown in FIG. 2C, both Klenow and Klenow exo—were detected at similar levels when compared to wild type DNA polymerase I, providing evidence that the DPE-PCR assay signal is derived from DNA polymerase-dependent extension and not intrinsic exonuclease activity.

In addition to using exonuclease free polymerases, we set out to further demonstrate that DPE-PCR assay signal is derived from DNA polymerase-dependent extension of the DNA substrate prior to qPCR. Since incorporation of dideoxy nucleotides is a well established method used for termination of DNA polymerase chain extension activities (19,20), we chose to substitute dCTP with dideoxyCTP (ddCTP) within our DNA polymerase extension reaction mix. The schematic shown in FIG. 2D reveals the first possible position within the substrate that ddCTP can be incorporated by DNA polymerase. If ddCTP is incorporated into this position, the extension product of Oligo 1 would be insufficient in length for subsequent detection by qPCR primer 1 (See FIG. 1 schematic). As shown in FIG. 2D, substitution of dCTP with ddCTP eliminates signal generated by DNA polymerase I, thus demonstrating that the DPE-PCR assay signal is dependent upon DNA polymerase extension of the substrate prior to qPCR. The presence of a low copy competitive internal amplification control confirms that qPCR was not inhibited by the presence of low amounts of ddCTP that are carried over from the DNA polymerase assay reagents.

In addition, a weak, but detectable signal was observed in the absence of input-DNA polymerase (No Input Control). Due to the exquisite sensitivity of the DPE-PCR assay, we have demonstrated that weak background noise signals can be attributed to “contaminant” DNA polymerase activity present in the DNA polymerase extension stock reagents prior to reaction assembly. Consequently, pre-treatment of the DNA polymerase extension reagents (see materials and methods section) is routinely performed and is sufficient to eliminate the contaminant DNA polymerase signal observed (See FIG. 2A for an example). Additionally, we have demonstrated that a major potential source of unwanted Taq-dependent signal could arise from the operator's failure to add active UDG to the qPCR mastermix. For example, intentional omission of UDG from the qPCR mastermix results in a high background signal derived from Taq-dependent extension of the DNA substrate (see FIG. 2B), however we have never observed high background signals (resulting from UDG failure) when UDG is added as described in the methods section. Another hypothesized source of increased background signal could be derived from DNA polymerase introduced by the operator during experimental setup. It is therefore recommended that, in the practice of the present invention, the operator exhibit good aseptic technique when preparing samples and reagents for the DNA polymerase extension and qPCR portions of the assay (see materials and methods section for contamination prevention recommendations). Considering the above, we feel it is very important that an NIC be run in parallel with each experiment to verify that the starting reagents are free of contamination and that UDG has been added to the qPCR mastermix.

Conclusion:

These data show an excellent linear relationship with a linear dynamic range of at least five orders of magnitude, with a lower limit of detection down around the 50 DNA polymerase molecule level. This example's data provides the foundation for development of a reliable and robust quantitative assay for DNA polymerase molecules, intact cells and the subcellular organelles that harbor these polymerases such as nuclei and mitochondria.

Methods:

S. aureus and E. coli cultures were grown to an OD₆₀₀ of 1.0±0.2 (approximately 1×10⁹ cfu/mL.) For each organism, 1 mL of culture was pelleted and washed three times in T.E. Bacterial suspensions were serially diluted in T.E., and 5 μL of each stock were added to bead mill lysis tubes containing 50 μL DNA polymerase extension reaction mixture (see above for composition). A titration curve of 1×10⁵ to 1×10° cfu/reaction was performed in triplicate for each organism, including triplicate reactions without bacterial suspension.

Bead mill lysis tubes are generated by pipetting 60 μL (wet volume) of 0.1 mm glass beads (Scientific Industries cat# SI-G01) using a 100 μL size Eppendorf tip and 50 μL (wet volume) of 0.5 mm glass beads (Scientific Industries cat# SI-BG05) using a modified 1000 μL size Eppendorf tip (To enable more reproducible and accurate dispensing of the 0.5 mm beads, the end of the 1000 μL size Eppendorf tip was cut to a 1 mm inner diameter using a sterile razor blade). Once a slurry of both size beads were dispensed into a 1.5 mL tube (with screw cap), the aqueous supernatant was subsequently aspirated using a sterile gel loading pipette tip attached to a vacuum source. After aspiration, tubes were capped and heat treated prior to use (see above heat treatment section).

After the addition of 5 μL bacterial stock, reaction tubes were bead milled for 6 min. at 2800 rpm using a digital Vortex Genie equipped with a disrupter head (Scientific Industries). Immediately after disruption, sample tubes were placed at 37° C. for 20 minutes. After the 20 minute incubation, sample tubes were transferred to 95° C. for 5 min. and removed to cool at room temperature. Sample tubes were then spun at 12 k×g for 30 seconds and 3 μL of each reaction were placed into the qPCR portion of the DPE-PCR assay. Five micro-liters of each bacterial stock was plated to obtain more accurate cfu input levels. Gene-specific PCR was also performed on the same lysates used for DNA polymerase detection.

### DNA Substrate Design and Preparation

The sequences of the DNA substrate were adapted from DNA oligos previously used to measure bacterial-derived ATP via T4 DNA ligase (18). Oligo 1 (5′-gccgatatcggacaacggccgaactgggaaggcgaga ctgaccgaccgataagctagaacagagagacaacaac-3′) and Oligo 2 (5′-uaggcgucggugacaaacggccagcguuguugu cucu[dideoxyCytidine]-3′) were synthesized by Integrated DNA Technologies (Coralville, Iowa). The “u” in Oligo 2 represents deoxyUridine. DideoxyCytidine (ddC) was included as the last base on the 3′ end of Oligo 2 to block DNA polymerase-mediated extension (see FIG. 1 schematic). First, lyophilized Oligo 1 and Oligo 2 were resuspended to a final concentration of 100 μM in sterile Tris-EDTA (T.E.) pH 8.0 (Ambion). Routine pre-annealing of the substrate was performed as follows. To begin, 100 μL of Oligol (100 μM stock) and 100 μL of Oligo 2 (100 μM stock) were added to 800 μL of annealing buffer (200 mM Tris, 100 mM Potassium chloride and 0.1 mM EDTA) pH 8.45 resulting in a 1 mL mixture of Oligo 1 and Oligo 2 each at 10 μM. One hundred micro-liter aliquots of the 10 μM oligo mixture was dispensed into thin-walled 0.2 mL PCR tubes, capped, placed into a GeneAmp® 9700 thermocycler (Applied Biosystems) and the following pre-annealing program was performed: 95° C. for 2 minutes, ramp at default speed to 25° C. and incubate for 5 minutes, ramp at default speed to 4° C. A substrate dilution buffer was prepared by diluting oligo annealing buffer (described above) 1:10 in sterile water (Ambion, cat#AM9932). The pre-annealed DNA substrate was subsequently diluted to a final concentration of 0.01 μM (10× stock) in oligo dilution buffer, aliquoted and stored at −20° C.

### Quantitative PCR Primers, Probes and Competitive Internal Control Design

The DPE-PCR primers described here were previously used to amplify a DNA substrate modified by T4 DNA ligase (18) and are as follows: Forward primer (5′-ggacaacggccgaactgggaaggcg-3′), Reverse primer (5′-taggcgtcggtgacaaacggccagc-3′). The detection probe used in this study was (5′ FAM-actgaccgaccgataagctagaacagagag-IABk-FQ 3′). As a tool to monitor qPCR inhibition, a competitive internal control was generated and contains the following sequence (5′-gccgatatcggacaacgg ccgaactgggaaggcgagatcagcaggccacacgttaaagacagagagacaacaacgctggccgtttgtcaccgacgccta-3′). The internal control sequence was synthesized and cloned as a “minigene” by Integrated DNA Technologies (Coralville, Iowa). Upon receipt, the internal control minigene plasmid was linearized using the restriction enzyme PvuI (New England Biolabs) and re-purified using a PCR cleanup column (Qiagen). The purified internal control was quantified using a Nanodrop spectrophotometer (Thermo Scientific, ND-1000), diluted to the desired concentration in T.E. and stored a −20° C. A probe, specific for the internal control DNA, was synthesized by Integrated DNA Technologies (5′ TX615-atcagcaggccacacgtt aaagaca-IAbRQSp 3′). A detailed schematic containing the relative positioning of the primers/probes within the substrate/competitive Internal Control can also be found in FIG. 1.

DNA Polymerase Extension Reaction Conditions

DNA Pol I (NEB cat# MO209L), Klenow (NEB cat# MO210S) and Klenow exo(−) (NEB cat# MO212S) were diluted to the indicated U/μL stock in sterile T.E. pH 8.0. To begin, 2 μL of DNA polymerase stock at each concentration were placed into a 50 μL DNA polymerase extension reaction mixture containing the following components: 50 μM dNTP, 20 mM Tris pH 8.0, 10 mM Ammonium sulfate, 10 mM Potassium chloride, 2 mM Magnesium sulfate, 1% BSA, 0.1% Triton X-100, 0.1% Tween 20, and 0.001 μM pre-annealed DNA substrate (described above. Two micro-liters of T.E. (without DNA polymerase) was routinely added to an additional tube containing complete DNA polymerase extension reaction mixture and is referred to as a “No Input Control” (NIC). Reactions containing DNA polymerase (or No Input Controls) were vortexed briefly and placed at 37° C. for 20 minutes. After 20 minutes, 3 μL of each reactions containing purified DNA polymerase were immediately placed into a qPCR reaction (see below for qPCR conditions).

### 2′,3′-Dideoxycytidine-5′-Triphosphate(ddCTP) Based, Dideoxy Chain Termination Experiments

Termination of Purified DNA Polymerase Extension Activity with ddCTP:

DNA polymerase extension reactions were prepared as described above with a 50 μM [dATP, dGTP, dTTP] mixture supplemented with either 50 μM dCTP or 50 μM ddCTP (Affymetrix #77332.) 50 μL DNA polymerase extension reactions with a 50 μM [dATP, dGTP, dTTP] mixture, supplemented with either dCTP or ddCTP, were spiked with 2 μL of a 1×10⁻⁹ U/μL stock of DNA polymerase I (New England Biolabs # MO209). Triplicate reactions were incubated at 37° C. for 20 minutes and 3 μL of each reaction were subsequently placed into qPCR.

Heat Treatment of DNA Polymerase Extension Reaction Components

Prior to usage, DNA polymerase extension reaction reagent stocks (minus DNA substrate) were heat treated as follows: 10×dNTP mixture [500 μM dATP, dCTP, dGTP, dTTP] was heated at 90° C. for 30 minutes. 10× core reaction mix [200 mM Tris pH 8.0, 100 mM Ammonium sulfate, 100 mM Potassium chloride, 20 mM Magnesium sulfate] was heated at 90° C. for 30 minutes. 1.43×BSA/Detergent mix [1.43% BSA, 0.143% Triton X-100, 0.143% Tween 20] was heated at 75° C. for 45 minutes. Substrate annealing buffer (200 mM Tris, 100 mM Potassium chloride and 0.1 mM EDTA) pH 8.45 was heated at 90° C. for 30 minutes. Bead mill tubes were heated at 95° C. for 20 minutes.

### Quantitative PCR Composition and Thermocycling Parameters

Each 30 μL qPCR reaction contained: 1× LightCycler 480 Master Mix (from 2× stock, Roche cat#04707494001), 333 nM of forward and reverse primers, 166 nM detection probe (FAM), 166 nM internal control probe (TxRed), 1.2 U of Uracil DNA Glycosylase (abbreviated hereafter as UDG, Bioline cat# BIO-20744) and 40 copies of the competitive Internal Control DNA (described above). Three micro-liters of each DNA polymerase extension reaction (from purified DNA polymerase or microbial cell lysates) were added to 27 μL of qPCR master mix and a two-step thermocyling protocol was run on a SmartCycler (Cepheid, Sunnyvale Calif.) as follows: Initial incubation of 40° C. for 10 minutes and 50° C. for 10 minutes and at 95° C. for 5 minutes (to activate Taq and complete UDG-mediated DNA backbone hydrolysis of Oligo 2), followed by 45 cycles of 5 s denaturation at 95° C. and 20 s annealing/extension at 65° C. Cycle threshold (Ct) values were generated automatically by the SmartCycler software using 2^{nd} derivative analysis of the emerging qPCR curves.

### Example 2

### Sensitive, Quantitative and Universal Detection of Microbes Via Measurement of Endogenous DNA Polymerase Extension Activity Directly from Cell Bead Mill Lysates

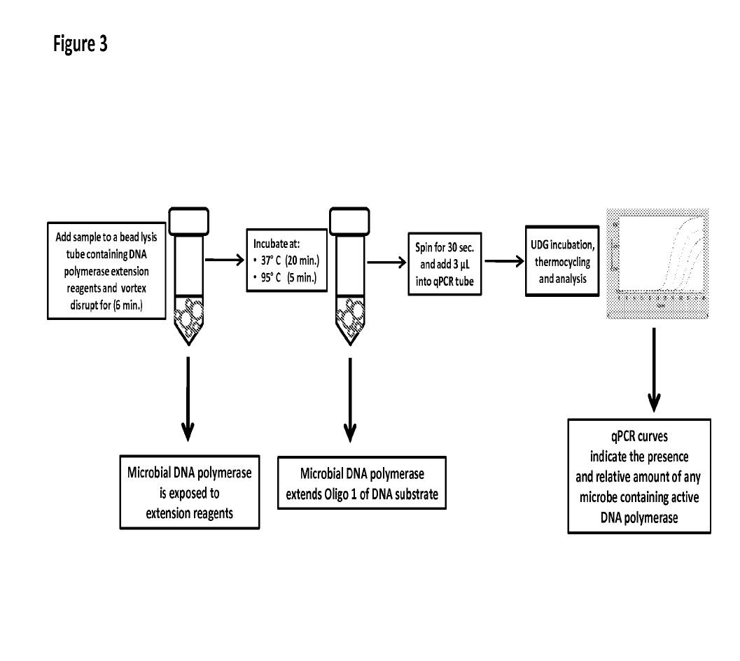

In addition to detecting purified polymerase activity a simple, sensitive and universal method that measures microbial-derived DNA polymerase activity would be highly desirable. For instance, measurement of DNA polymerase extension activity could be used to screen environmental or biological samples for the presence of any microorganism harboring active DNA polymerase. To this end, we developed a simple method that couples microbial lysis to our DPE-PCR assay. As shown in FIG. 3, a liquid sample known to contain, or suspected of containing, microbes is added to a bead mill lysis tube, disrupted and immediately transitioned into the DPE-PCR assay. We chose one gram negative bacteria (E. coli) and one gram positive bacteria (S. aureus) to demonstrate the ability of our assay to measure microbial-derived DNA polymerase extension activity in crude cellular lysates. As shown in FIG. 4A, when linked with bead mill lysis, the DPE-PCR assay is capable of detecting a wide dynamic range of input E. coli, down to and below 10 colony forming units (cfu) per lysis tube. Linear regression analysis of E. coli detection was also performed down to 10 cfu of input bacteria and showed a strong positive linear correlation between input cfu and DNA polymerase extension activity signal as indicated by an R² value of 0.999 (FIG. 4B). Colony count plating and E. coli-gene specific qPCR (gsPCR) were run in parallel, confirming both the input level of cfu per reaction and the ability to monitor intact genomic DNA from the exact same lysates. DNA polymerase extension activity from S. aureus lysates was detected to a similar input level (FIG. 4C). S. aureus detection was plotted down to 10 cfu of input bacteria and also showed a strong linear correlation between input cfu and DNA polymerase extension activity signal (R²=0.999, FIG. 4D). Colony count plating and gsPCR were performed in parallel to confirm the amount of S. aureus present in each bead lysis tube, as well as the presence of directly analyzable genomic DNA.

Elimination of DPE-PCR Detection of Microbes Via ddCTP Substitution

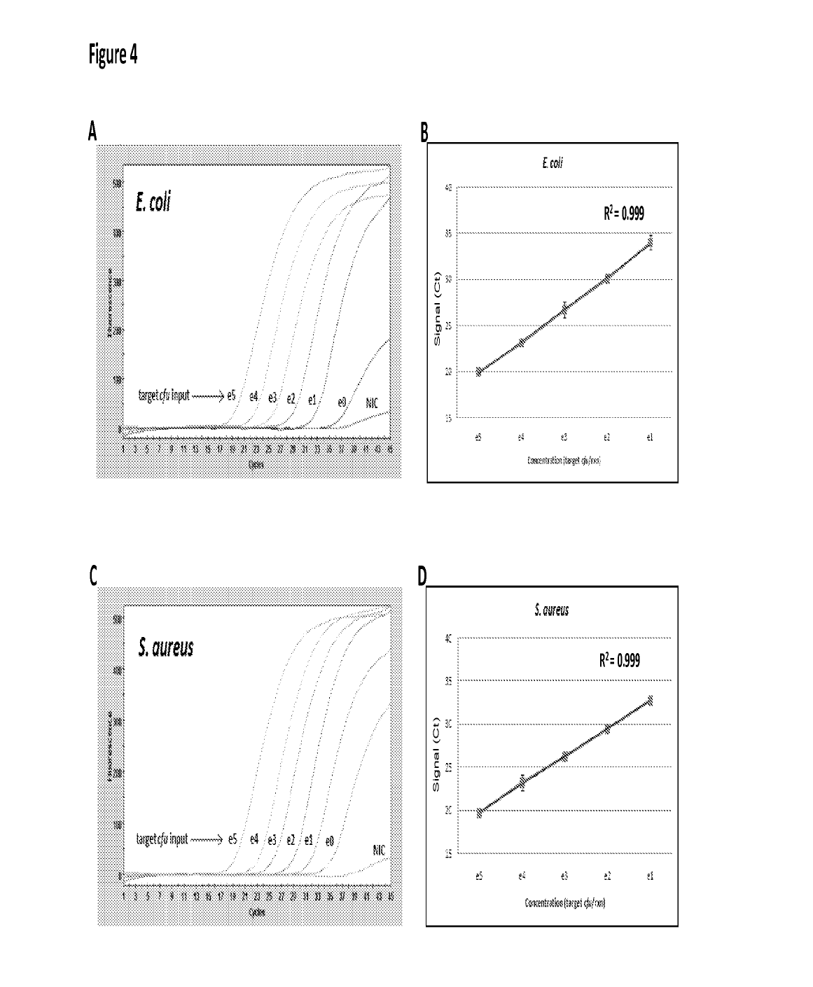

As previously shown in FIG. 2D, substitution of dCTP with ddCTP in the DNA polymerase extension reaction mix represents a powerful tool for blocking extension of Oligo 1 within our assay. To demonstrate that the signal derived from bacterial spikes was dependent upon their DNA polymerase extension activity, and not the other endogenous bacterial enzyme activities present in the lysates, we set up an experiment to compare DPE-PCR signals obtained from E. coli and S. aureus using a standard DNA polymerase reaction mix containing (dATP, dTTP, dGTP, dCTP) versus a reaction mix containing (dATP, dTTP, dGTP, ddCTP). As shown in FIGS. 5A and 5B, when compared to the standard reaction mix, substitution of ddCTP blocked the generation of signal derived from E. coli, S. aureus cfu spikes. Together, the data presented strongly support the claim that the DPE-PCR assay is specifically detecting microbial DNA polymerase extension activity and signal is not derived from substrate modification via enzymatic activities other than DNA polymerase.

### Table 1: The Following Table Shows Data Representative of Sensitive and Linear Detection of 17 Additional Clinically Relevant Microbial Species, Using Preferred Embodiments of the Assay of the Present Invention.

TABLE 1

| | Lower Limit Detected by | |
|---|---|---|
| Bacterial panel | DPE-PCR | R² (1e4-1e1 cfu) |
| Klebsiella pneumoniae | <10 | 0.9957 |
| Pseudomonas aeruginosa | <10 | 0.9860 |
| Enterobacter cloacae | <10 | 0.9995 |
| Acinetobacter baumannii | <10 | 0.9980 |
| Haemophilus influenzae | <10 | 0.9996 |
| Serratia marcescens | <10 | 0.9956 |
| Enterococcus faecalis | <10 | 0.9963 |
| Enterococcus faecium | <10 | 0.9899 |
| Streptococcus pyogenes | <10 | 0.9945 |
| Streptococcus agalactiae | <10 | 0.9969 |
| Streptococcus pneumoniae | <10 | 0.9999 |
| Staphylococcus epidermidis | <10 | 0.9990 |

| | Lower Limit Detected by | |
|---|---|---|
| Candida panel | DPE-PCR | R² (1e5-1e3 cfu) |
| Candida albicans | ≈20 | 0.9945 |
| Candida tropicalis | ≈20 | 0.9969 |
| Candida glabrata | ≈40 | 0.9111 |
| Candida parapsilosis | ≈20 | 0.9950 |
| Candida krusei | ≈15 | 0.9868 |

Conclusions:

In summary, in accordance with the present invention we have developed a novel, highly sensitive, quantitative and rapid DPE-PCR assay that can be used to enumerate prokaryotic cells when presenting a purified or selected cell type. These data show an excellent linear relationship with a linear dynamic range of at least five orders of magnitude. We have demonstrated the ability of DPE-PCR to reproducibly measure DNA polymerase extension activity from less than 10 cfu of bacteria via coupling to bead lysis. We have also demonstrated the potential for the DPE-PCR assay of the invention to universally detect microbes by testing a panel of microorganisms comprised of gram-negative bacteria, gram-positive bacteria and Candida species. Furthermore, it has been shown that the DPE-PCR assay can be used to assess bacterial cell viability was provided via the reproducibly strong correlation between DNA polymerase extension activity and proliferation as indicated by the presence of cfu. Considering the data presented herein, we believe that the ETGA methodology exemplified by the DPE-PCR assay of the present invention has the potential to become a useful quantitative tool for a wide range of testing applications within pharmaceutical, environmental, food and clinical settings.

### Example 3

### Intact Human Platelet Concentrates Contain High Levels of DNA Polymerase Extension Activity

### Objective:

To test for the presence of detectable DNA polymerase extension activity using the ETGA assay of the invention, activity from crude bead mill lysates from viable human Platelet Concentrates (PC) collected via three different methodologies, Whole Blood Derived, Apheresis Non-Leukoreduced, Apheresis Leukoreduced.

### Methods:

### Removal of Platelets

Remove platelet bag from incubator

Add a blue ‘slide pinch clamp’ to the tubing, adjacent to the tubing neck.

Suspend bag from hood ceiling using large paper clip

Flame-sterilize scissors/clippers and wipe the end of the tubing being used for removal with an alcohol wipe

Position a 15 ml conical (for ‘purging’ the platelet volume trapped in tubing) below the tube

Use sterile clippers to cut the tubing near its closed end.

Slowly slide the clamp to the ‘open’ position and allow 5 ml of platelets to flow into the 15 ml conical vial, and slide to the ‘closed’ position.

Position a second 15 ml conical below the tubing

Slowly slide the clamp to the ‘open’ position and allow 5 ml of platelets to flow into the 15 ml conical vial, and slide to the ‘closed’ position.

Place surgical clamp near the open end of the tubing and wipe with an alcohol pad to remove drips from the open end.

### Pre-ETGA Preparation

Don fresh gloves and clean with IPA

Remove the following reagent aliquots from freezer and thaw at the indicated temperatures:
5×/dNTP [blue cap]-Room temperature (For 2′,3′-Dideoxycytidine-5′-Triphosphate (ddCTP) DNA polymerase extension termination experiments, a fresh dNTP mix was assembled with ddCTP instead of dCTP).
Substrate [white cap]-Room temperature
BSA/Detergent [green cap]-Room temperature
qPCR oligo mix [orange cap]-Room temperature
Roche Probes Master Mix [orange cap]-Room temperature

### ETGA Sample Preparation

Add 0.5 ml PC to a separate empty tube and designate as ‘Non-Lysed PC’ and cap

Blood Agar Bacterial Culture Plated with 100 uL of PC to verify sterility (In most cases, 8 mL of PC were also inoculated into both aerobic and anaerobic blood culture bottles to verify sterility of the PC unit). Plates incubated at 37 for 48 hrs, colony number recorded. Blood culture bottles inoculated were incubated in automated incubator for 5 days.

Spin at 8000×g for 3 min

Pour off supernatant and invert tube onto a plastic-backed lab wipe (Thomas Cat#2904N90) (hold for 3 seconds)

Add 0.6 ml of sterile saline to the Non-Lysed control and pipette up and down to mix, and simultaneously transfer to pre-labelled beadmill tube

Centrifuge at 8000×g for 3 min.

Carefully remove supernatant using a 1 ml pipette. (it is important to remove as much residual liquid as possible without excessive disruption of the bead bed.)

Assemble lysis mix as follows:

Lysis Mix Setup—Enough for n=10×50 ul Reactions (Add Reagents in Below-Listed Order):
After tubes are thawed, vortex and pulse spin to collect contents
Add 100 uL of 5×/dNTP mix (blue cap) to BSA/Detergent mix (green cap)
Add 50 uL of substrate (white cap) to BSA/Detergent mix (green cap)
Cap BSA/Detergent mix (green cap) tube and vortex to mix
Pulse spin to collect contents
Add 50 ul of Lysis Mix to samples and controls

### Cell Lysis:

Add 50 ul lysis mix to each beadmill tube

Place beadmill tubes into disrupter head and vortex at 2800 rpm for 6 min

Add 5 ul of DNA polymerase (the pre-diluted PC stock) to the DNA pol control tube and briefly vortex

### Enzymatic Modification of Substrate:

Place each tube at 37° C. for 20 min.

Transfer each tube to 95° C. heat block for 5 min.

Assemble PCR master mix (×2) during the 5 min. incubation.
Add 150 ul of Roche Probes Mastermix to the oligomix tube
Add 12 ul of UNG to the oligomix tube
Vortex and pulse spin to collect

After heating at 95° C., let tubes sit at room temp for 1 min

Add 27.2 μl PCR mmx to each pre-labelled SMART cycler tube (Cepheid Part#900-0003)

Spin beadmill tubes for 30 seconds at 12000×g

Add 4 μl of lysate to PCR reaction tube

Run PCR on SMART Cycler using PolMA SLBN assay definition:

| | |
|---|---|
| 1 cycle | 40° C. 10 minutes |
| | 50° C. 10 minutes |
| | 95° C. 5 minutes |
| 45 cycles | 95° C. 5 seconds |
| | 65° C. 20 seconds |

### Results:

1. All units tested were negative for the presence of bacteria by incubation for either plating, blood culture, or both.
2. All 3 methods for preparing PC yielded robust DNA polymerase signals after intact PC cell membranes were disrupted via bead mill liberating cell contents.
3. For all 3 methods DNA polymerase was proven to account for all the measured extension activity via ddCTP chain termination experiments.
   See FIG. 6 for graphical data representations of the above and of DNA polymerase Specificity Experiment via ddCTP extension termination.
   Note: ETGA analysis of Intact PC that were not chemically lysed/denatured “Non-lysed PC” performed prior to subsequent bead mill based disruption of plasma membranes forming a crude cell lysate containing native enzyme activities such as DNA polymerase.

### Conclusions:

The ddCTP experiment proves that these human PC derived signals are dependent upon DNA polymerase extension activity. Thus, ETGA assay detects high levels of DNA polymerase signal from sterile intact platelet concentrates following bead mill membrane disruption regardless of the method of PC preparation. This mammalian PC ETGA signal is expected to be predominantly from platelet derived mitochondrial gamma-DNA polymerase activity as platelets are devoid of nuclei. However in PC, minor polymerase signal contribution cannot be ruled out from contaminating nucleated white blood cells. Based on the literature, it is reasonably expected that all mammalian blood cell types, except for red blood cells which lack both nucleus and mitochondria, will produce strong DNA polymerase signals. One skilled in the art will appreciate that it is further expected that any mammalian cell containing a nucleus or mitochondria is a candidate for detection and quantification via this novel assay of the present invention.

### Example 4

### Measurement of DNA Polymerase Extension Activity as Sensitive, Quantitative Indicator of Human Cell Culture Cell Number and Viability

Objective: To determine if ETGA can detect DNA polymerase extension activity from in vitro cultured Hep2 cells.

### Methods:

Obtained a confluent T75 flask of Hep2 cells.
Harvested cells by washing flask with PBS, adding trypsin solution and quenched with media.
Transfer the contents from the flask (13 mL) to a 15 mL conical vial.
From the 15 mL vial, I removed 2×1 mL aliquots of the cell suspension and washed 3×with PBS. (spun @ 6 k×rpm for 2 min. each during wash)
Resuspend the final pellet in 1 mL PBS.
Make 1:5 dilutions of this stock in PBS and performed cell counts using the hemacytometer.
Add 5 uL of n=5 of the diluted cell suspensions (and a non-spiked control) to beadmill tubes containing 50 ul of DPE mix.

### Organism Lysis:

Add 50 ul lysis mix to each beadmill tube

Place beadmill tubes into disrupter head and vortex at 2800 rpm for 6 min

### Enzymatic Modification of Substrate:

Place each tube at 37° C. for 20 min.

Transfer each tube to 95° C. heat block for 5 min.

Assemble PCR master mix during the 5 min. incubation.
Add 150 ul of Roche Probes Mastermix to the oligomix tube
Add 12 ul of UNG to the oligomix tube
Vortex and pulse spin to collect

After heating at 95° C., let tubes sit at room temp for 1 min

Add 27.2 μl PCR mmx to each pre-labelled SMART cycler tube (Cepheid Part#900-0003)

Spin beadmill tubes for 30 seconds at 12000×g

Add 4 μl of lysate to PCR reaction tube

Run PCR on SMART Cycler using PolMA SLBN assay definition:

| | |
|---|---|
| 1 cycle | 40° C. 10 minutes |
| | 50° C. 10 minutes |
| | 95° C. 5 minutes |
| 45 cycles | 95° C. 5 seconds |
| | 65° C. 20 seconds |

### Results:

### Cell Counts

Level 1=1×10⁶ cells
Level 2=2×10⁵ cells
Level 3=4×10⁴ cells
Level 4=8×10³ cells
Level 5=1.6×10³ cells

### Conclusions:

ETGA assay methods performed in accordance with the present invention are capable of detection of DNA polymerase extension activity associated with in vitro cultured Hep2 cells. It is reasonably assumed that this assay method can detect any DNA polymerase from any intact viable cell and or their polymerase harboring subcellular organelles such as nuclei, mitochondria etc.

### Example 5

### Example

### Reverse Transcriptase (RT) Detection Assay

### Objective:

To perform an experiment aimed at assessing the ability to detect reverse transcriptase activity using a DNA (S1)/RNA(AS) substrate within our basic DPE-PCR assay system. This embodiment of the ETGA assay technology of the invention could enable applications such as, but not limited to: screening of reverse transcriptase inhibitors for the drug development industry and detection of viral particles in biological samples (HIV).

### Methods:

Pre-annealed standard S1 (DNA) and an RNA version of the SASext-oligonucleotide according to procedure described in DNA-oligonucleotide substrate preparation.
Dilute pre-annealed oligonucleotide extension substrate in a 1:10 dilution of 10×RT buffer to a final concentration of 0.01 uM.
   Assemble the following reaction mix using reagents supplied with the SSIII kit (Invitrogen):

### Add Per Reaction

| | |
|---|---|
| 5 ul | substrate |
| 2 ul | 10X RT buffer |
| 4 ul | MgCl2 (25 mM) |
| 2 ul | DTT (0.1M) |
| 0.5 ul | RNase OUT |
| 1 ul | dNTP mix |
| 3.5 ul | Water |
| 18 ul | per reaction tube |
| +2 ul | of RT dilutions (Made in T.E.) |
| 20 ul | |

After adding 2 ul of each RT dilution (or 2 ul of T.E. for reagent control) incubate at 37° C. for 20 minutes
Add 3 ul of reaction to PCR reaction (not containing UNG) and cycle without UNG pre-incubation steps.

### Reaction ID

1. 1E⁻² dilution of RT

2. 1E⁻⁴ dilution of RT

3. 1E⁻⁶ dilution of RT

4. 1E⁻⁸ dilution of RT

5. 1E⁻¹° dilution of RT

6. 1E⁻⁸ dilution of DNA Pol I (*does contain some intrinsic RT activity)

7. T.E.

8. T.E.

9. PCR-Blank (T.E.)

### Conclusions:

Detection of reverse transcriptase activity using only a simple RNA-oligonucleotide in place of the DNA-AS-oligonucleotide has been successfully demonstrated. Reagent background (T.E. only) is completely negative (even without UNG within the PCR), demonstrating that Taq DNA polymerase does not extend DNA:RNA-hybrid primer extension substrate.

### Example 6

### Example

### Human Immunodeficiency Virus (HIV) Reverse Transcriptase Detection Assay

### Objective:

To perform an experiment aimed at assessing the ability to detect recombinant HIV reverse transcriptase activity using a DNA (S1)/RNA(AS) substrate within the ETGA assay system of the present invention.

### Methods:

Recombinant HIV RT (Calbiochem cat#382129)
   Assemble the following reaction mix using reagents supplied with the SSIII kit (Invitrogen):

### Add Per Reaction

| | |
|---|---|
| 5 ul | Pre-annealed RNA/DNA substrate (0.01 uM) |
| 2 ul | 10X RT buffer |
| 4 ul | MgCl2 (25 mM) |
| 2 ul | DTT (0.1M) |
| 0.5 ul | RNase OUT |
| 1 ul | dNTP mix |
| 3.5 ul | Water |
| 18 ul | per reaction tube |
| +2 ul | of RT dilutions (Made in T.E.) |
| 20 ul | |

After adding 2 ul of each RT dilution (or 2 ul of T.E. for reagent control) incubate at 37° C. for 20 minutes
Add 3 ul of reaction to PCR reaction (not containing UNG) and cycle without UNG pre-incubation steps.

### Reaction ID

10. 1E⁻² dilution of HIV-RT
11. 1E⁻⁴ dilution of HIV-RT
12. 1E⁻⁶ dilution of HIV-RT
13. 1E⁻⁸ dilution of HIV-RT
14. 1E⁻¹° dilution of HIV-RT
15. 1E⁻⁴ dilution of Superscript III RT enzyme (Pos control)
16. T.E.
17. T.E.
18. PCR-Blank (T.E.)

### Conclusions:

Detection of HIV reverse transcriptase activity using only a simple AS-oligo substitution RNA-oligonucleotide has been demonstrated as enabled by the novel assay of the present invention. Reagent background (T.E. only) is completely negative (even without UNG within the PCR), again verifying that Taq DNA polymerase does not recognize this DNA:RNA-hybrid primer extension substrate. This example demonstrates that HIV reverse transcriptase can be substituted in place of DNA polymerase for detection and quantification of RT enzyme activity and or any cell or subcellular organelle component that harbors active HIV RT or a viable viroid.

The contents of all references, patents and published patent applications cited throughout this application, are incorporated herein by reference to the same extent as if each individual publication, patent or patent application was specifically and individually indicated to be incorporated by reference.

The foregoing detailed description has been given for clearness of understanding only and no unnecessary limitations should be inferred therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

### REFERENCES

1. Rothwell, P. J and Waksman, G. (2005) Structure and mechanism of DNA polymerases. Advan. Prot. Chem. 71 401-440.
2. Joyce, C. M. and Steitz, T. A. (1994) Function and structure relationships in DNA polymerases. Annu. Rev. Biochem. 63, 777-822.
3. Mar Albà, M. (2001) Replicative DNA polymerases. Genome Biol. 2, 3002.1-3002.4.
4. Lehman, I., Bessman, M., Simms, E. and Kornberg, A. (1958) Enzymatic synthesis of deoxyribonucleic acid. I. preparation of substrates and partial purification of an enzyme from Escherichia coli. J. Biol. Chem. 233, 163-170.
5. Hamilton, S. C., Farchaus, J. W., and Davis, M. C. (2001) DNA polymerases as engines for biotechnology. BioTechniques 31, 370-383.
6. Tarantino, P. M., Zhi, C., Wright, G. E., and Brown, N. C. (1999) Inhibitors of DNA polymerase III as novel antimicrobial agents and gram-positive bacteria. Antimicrob. Agents Chemother. 43, 1982-1987.
7. Kuhl, A., Svenstrup, N., Ladel, C., Otteneder, M., Binas, A., Schiffer, G., Brands, M., Lampe, T., Ziegelbauer, K., Rühsamen-Waigmann, H., Haebich, D. and Ehlert, K. (2005) Biological characterization of novel inhibitors of the gram-positive DNA polymerase IIIC enzyme. Antimicrob. Agents Chemother. 49, 987-995.
8. Lange, S. S., Takata, K. and Wood, R. D. (2011) DNA polymerases and cancer. Nat. Rev. Cancer 11, 96-110.
9. Martin, S. A., McCabe, N., Mullarkey, M., Cummins, R., Burgess, D. J., Nakabeppu, Y., Oka, S., Kay, E., Lord, C. J. and Ashworth, A. (2010) DNA polymerases as potential therapeutic targets for cancers deficient in the DNA mismatch repair proteins MSH2 or MLH1. Cancer Cell 17, 235-248.
10. Naviaux, R. K., Markusic, D., Barshop, B. A., Nyhan, W. L. and Haas, R. H. (1999) Sensitive assay for mitochondrial DNA polymerase γ. Clin. Chem. 45, 1725-1733.
11. Richardson, C. C., Schildkraut, C. L., Vasken Aposhian, H. and Kornberg, A. (1964) Enzymatic synthesis of deoxyribonucleic acid. J. Biol. Chem. 239, 222-232.
12. Griep, M. A. (1995) Flurescence recovery assay: A continuous assay for processive DNA polymerase applied specifically to DNA polymerase III holoenzyme. Anal. Biochem. 232, 180-189.
13. Seville, M., West A. B., Cull, M. G. and McHenry, C. S. (1996) Fluorometric assay for DNA polymerases and reverse transcriptase. Biotechniques 21, 664, 668, 670, 672.
14. Tveit, H. and Kristensen, T. (2001) Fluorescence-based DNA polymerase assay. Anal. Biochem. 289, 96-98.
15. Yu, Liming, Hu, G. and Howells, L. (2002) Fluorescence-based, high-throughput DNA polymerase assay. Biotechniques 33, 938-941.
16. Ma, C., Tang, Z., Wang, K., Tan, W., Li, J., Li, W., Li, Z., Yang, X., Li, H. and Liu, L. (2006) Real-time monitoring of DNA polymerase activity using molecular beacon. Anal. Biochem. 353, 141-143.
17. Luo, X. and Hsing I. (2011) Immobilization-free electrochemical DNA polymerase assay. Electroanalysis 23, 923-926.
18. Banin, S., Wilson, S. and Stanley C. (2007) The LiMA technology: measurement of ATP on a nucleic acid testing platform. Clin Chem 53, 2034-2036.
19. Toji, L. and Cohen, S. S. (1969) The enzymatic termination of polydeoxynucleotides by 2′3′-dideoxyadonsine triphosphate. Proc. Natl. Acad. Sci. USA 63, 871-877.
20. Sanger, F., Nicklen, S. and Coulson, R. (1977) DNA sequencing with chain-terminating inhibitors. Proc. Natl. Acad. Sci. USA 74, 5463-5467.
21. Davey, H. M. (2011) Life, death, and in-between: Meanings and methods in microbiology. Appl. Environ. Microbiol. 77, 5571-5576.
22. Rappe, M. S. and Giovannoni, S. J. (2003) The uncultured microbial majority. Annu. Rev. Microbiol. 57, 369-394.
23. Riley, B. (2004) Rapid microbiology methods in the pharmaceutical industry. Amer. Pharm. Rev. 1-4.
24. Keer, J. T. and Birch, L. (2003) Molecular methods for the assessment of bacterial viability. J. Microbio. Meth. 53, 175-183.
25. Masters, C., Shallcross, J. and Mackey, B. (1994) Effect of stress treatments on the detection of Listeria monocytogenes and enterotoxigenic Escherichia coli by the polymerase chain reaction. J. Appl. Bacteriol. 77, 73-79.
26. Sheridan, G. E. C., Masters, C. I., Shallcross, J. A. and Mackey, B. M. (1998) Detection of mRNA by reverse transcription-PCR as an indicator of viability in Escherichia coli cells. Appl. Environ. Microbiol. 64, 1313-1318.

What is claimed is:

1. A method for detecting polymerase activity in a sample, as an indicator of the presence of a viable cell or subcellular organelle containing an active polymerase, which method comprises the steps of:
(a) contacting the sample with a nucleic acid molecule which acts as a substrate for polymerase activity in the sample;
(b) incubating the contacted sample under conditions suitable for polymerase activity; and
(c) determining the presence (and/or the quantitative amount) of a nucleic acid molecule resulting from the action of the active polymerase on the substrate nucleic acid molecule, thereby to indicate the presence of the viable cell or subcellular organelle or total polymerase activity in the sample.

2. The method of claim 1, wherein the polymerase is a DNA polymerase.

3. The method of claim 1, wherein the polymerase is either an RNA polymerase.

4. The method of claim 1, wherein the viable cell or subcellular organelle is an intact viable cell or subcellular organelle in the sample.

5. The method of claim 4, wherein the intact viable cell or subcellular organelle is one in which the nucleic acid polymerase gene and its translated active protein polymerase is essential for viability of the viable cell or nucleic acid molecule capable of acting as a substrate for polymerase activity organelle.

6. The method of claim 1, wherein the nucleic acid molecule which acts as a substrate for polymerase activity is immobilized.

7. The method of claim 1, wherein the sample is prepared by using a differential cell lysis preparation method, thereby allowing only the polymerase activity from the viable cell or subcellular organelle to modify the substrate for polymerase activity.

8. The method of claim 1, wherein the sample is prepared from crude cell lysates or purified cell fractions.

9. The method of claim 8, wherein the method further comprises the step of conducting a viable cell or subcellular organelle genome or transcriptome sequence analysis.

10. The method of claim 9, wherein the sequence analysis is performed using a single sample preparation.

11. The method of claim 9, wherein the sequence analysis further comprises detecting agents having anti-viable cell or subcellular organelle or anti-polymerase activity useful in the diagnosis and management of pathological conditions in patients.

12. An assay kit comprising reagents useful in the method claimed in claim 1,
said kit being useful for screening for the presence or absence of viable cell or subcellular organelles in the sample and for providing diagnostic, prognostic, or patient management information.
